(19) United States
(12) Patent Application Publication
(10) Pub. No.: US 2018/0008539 A1
(43) Pub. Date: Jan. 11, 2018

(54) GASTRORETENTIVE EXTENDED RELEASE SUSPENSION COMPOSITIONS

(71) Applicant: SUN PHARMACEUTICAL INDUSTRIES LIMITED, Mumbai, Maharashtra (IN)

(72) Inventors: Romi Barat SINGH, Varanasi (IN); Ashish KUMAR, Jhajjar (IN); Paras P. JAIN, Amravati (IN); Suchitra KAUSHIK, Hardiwar (IN); Vinay KUMAR, Fatehabad (IN)

(73) Assignee: SUN PHARMACEUTICAL INDUSTRIES LIMITED, Mumbai, Maharashtra (IN)

(21) Appl. No.: 15/533,285

(22) PCT Filed: May 1, 2015
(86) PCT No.: PCT/IB2015/053208

§ 371 Date: Jun. 5, 2017

(30) Foreign Application Priority Data
Dec. 5, 2014 (IN) 3553/DEL/2014

(51) Int. Cl. A61K 9/00; A61K 9/50; A61K 31/155

(52) U.S. Cl. CPC A61K 9/0065; A61K 31/155; A61K 9/5089; A61K 9/0095; A61K 9/5047; A61K 9/5078

(57) ABSTRACT

The present invention relates to a gastroretentive extended release suspension composition, wherein the composition is characterized by having no substantial change in the in-vitro dissolution release profile upon storage for at least seven days. The invention also relates to processes for the preparation of said gastroretentive extended release suspension compositions.

### FIELD OF THE INVENTION

The present invention relates to a gastroretentive extended release suspension composition, wherein the composition is characterized by having no substantial change in the in-vitro dissolution release profile upon storage for at least seven days. The invention also relates to processes for the preparation of said gastroretentive extended release suspension compositions.

### BACKGROUND OF THE INVENTION

Extended release solid compositions are preferred dosage forms over immediate release solid compositions, especially for active ingredients showing fluctuations in the plasma concentration and for active ingredients having short half-lives. Extended release solid compositions can be in the form of tablets or capsules, wherein the release of the active ingredient is controlled by using a reservoir or a matrix system. However, extended release solid compositions suffer from certain drawbacks such as difficulty in swallowing, particularly for certain groups of patients, e.g., pediatrics and geriatrics, resulting in poor patient compliance. Further, high doses of active ingredient lead to large-sized compositions which aggravate this problem. Also, there remains a tendency to divide the extended release solid compositions such as tablets into small pieces in order to facilitate the administration, which may ultimately lead to inaccurate dosing and or dose dumping. In view of all this, extended release liquid compositions provide the best alternative over extended release solid compositions. Extended release liquid compositions are easy to administer, thereby leading to enhanced patient compliance.

Although extended release liquid compositions are advantageous, there remain some complexities involved in formulating such compositions. The important prerequisite of these compositions is to provide the desired extended release of the active ingredient throughout the shelf life, as irregular release may lead to sub-therapeutic or toxic effects.

The prior art discloses various approaches for the preparation of extended release liquid compositions.

U.S. Pat. No. 6,156,340 discloses a controlled release suspension comprising inert cores coated with an active ingredient, which were further coated with two layers of polymers having increasing permeability for water.

U.S. Pat. No. 7,906,145 discloses a sustained release suspension of microcapsules in an aqueous liquid phase, wherein each microcapsule comprises a core of an active ingredient and a film coating applied to the core which controls the modified release of the active ingredient in gastrointestinal fluids, comprising a film-forming polymer, a nitrogen-containing polymer, a plasticizer, and a surfactant/lubricant.

PCT Publication No. WO 2011/107855 discloses a ready to use sustained release oral suspension comprising inert pellets surrounded by a seal coating, an active ingredient layer surrounding the seal coated inert pellets, and a coating layer comprising a rate-controlling polymer surrounding the active ingredient layer.

PCT Publication No. WO 2011/077451 discloses a controlled release suspension comprising an active ingredient loaded core and a polymer dispersion comprising a controlled-release polymer, wherein said suspension has a duration of therapeutic effect for at least about 6 hours to about 30 hours after oral administration.

PCT Publication No. WO 2008/122993 discloses a suspension of an active ingredient containing microparticles with at least one coat of a pH-independent polymer.

PCT Publication No. WO 2012/063257 and U.S. Publication No. 2008/0118570 disclose controlled release suspensions employing ion-exchange resins.

The extended release liquid compositions as taught in the prior art are complicated and have a very short gastric residence time which may not be desirable for active ingredients which are absorbed through the upper part of the gastrointestinal tract and which are unstable in the intestine.

There remains a need in the art to formulate gastroretentive extended release liquid compositions which provide the consistent in-vitro dissolution release throughout the shelf life of the compositions and which are suitable for active ingredients which require prolonged gastric residence time.

The present invention provides such extended release liquid compositions which are based on a simplified technology and which provide a significant advance over the available extended release liquid compositions.

The gastroretentive extended release suspension compositions of the present invention are relatively simple, easy to manufacture, and functionally reproducible. Said gastroretentive extended release suspension compositions provide the desired extended release throughout the shelf life of the compositions. The scientists of the present invention have surprisingly discovered that a hypertonic condition generated in the suspension base affects the leaching of the active ingredient from the extended release coated cores into the suspension base. This hypertonic condition minimizes the leaching problem and thus provides substantially similar in-vitro extended release of the active ingredient throughout the shelf life of the composition. The use of a gel-forming agent and/or a gas-generating agent in the composition helps to achieve gastro-retention for active ingredients which are absorbed through the upper part of the gastrointestinal tract or which are unstable in the intestine.

### SUMMARY OF THE INVENTION

The present invention provides a gastroretentive extended release suspension composition, wherein the composition is characterized by having no substantial change in the in-vitro dissolution release profile upon storage for at least seven days. The gastroretentive extended release suspension compositions comprise active ingredients which are mainly absorbed in the stomach, which have higher solubility in the stomach than in the intestine, which are poorly absorbed or degraded in the intestine, or which act locally in the stomach. The invention also relates to processes for the preparation of the gastroretentive extended release suspension compositions.

The gastroretentive extended release suspension compositions of the present invention are easy to administer thereby leading to enhanced patient compliance. Also, said gastroretentive extended release suspension compositions are stable, easy to commercially manufacture, and provide reproducible bioavailability. Additionally, said gastroretentive extended release suspension compositions provide pleasant mouth feel thereby further enhancing the patient compliance.

### BRIEF DESCRIPTION OF THE DRAWINGS

FIG. 1 shows the in-vitro dissolution release on day 0 and day 30 of the gastroretentive extended release suspension composition prepared according to Example 1 upon storage at room temperature. The FIGURE also shows the in-vitro dissolution release on day 0 and day 30 of the gastroretentive extended release suspension composition (at room temperature) formed after reconstituting the powder stored for one month at accelerated conditions.

### DETAILED DESCRIPTION OF THE INVENTION

Dissolution release profiles described below and elsewhere in this application were typically determined by USP type II apparatus at 100 rpm, in 1000 mL of phosphate buffer with a pH 6.8, unless otherwise stated.

A first aspect of the present invention provides a gastroretentive extended release suspension composition comprising an active ingredient and a suspension base, wherein the composition is characterized by having no substantial change in the in-vitro dissolution release profile upon storage for at least seven days.

According to one embodiment of the above aspect, the active ingredient is in the form of multiple cores coated with a release-controlling polymer.

According to another embodiment of the above aspect, the gastroretentive extended release suspension composition is characterized by having an osmolality ratio of at least about 1.

According to another embodiment of the above aspect, the suspension base is responsible for creating a hypertonic environment.

According to another embodiment of the above aspect, the suspension base comprises an osmogent.

According to another embodiment of the above aspect, the suspension base comprises a gel-forming agent.

According to another embodiment of the above aspect, the suspension base comprises a gas-generating agent.

According to another embodiment of the above aspect, the suspension base comprises a gel-forming agent and a gas-generating agent.

According to another embodiment of the above aspect, the gastroretentive extended release suspension composition is in the form of a suspension or a reconstituted powder for suspension.

According to another embodiment of the above aspect, the release-controlling polymer is selected from the group comprising a pH-dependent polymer, a pH-independent polymer, or mixtures thereof.

According to another embodiment of the above aspect, the core is in the form of a bead, a pellet, a granule, a spheroid, or the like.

According to another embodiment of the above aspect, the active ingredient is layered onto an inert particle to form the core.

A second aspect of the present invention provides a process for the preparation of a gastroretentive extended release suspension composition, wherein the process comprises the steps of:
(i) preparing cores comprising an active ingredient and one or more pharmaceutically acceptable excipients;
(ii) dissolving/dispersing a release-controlling polymer and one or more pharmaceutically acceptable coating additives in a suitable solvent;
(iii) applying the coating composition of step (ii) over the cores of step (i);
(iv) dissolving/dispersing one or more osmogents, one or more gel-forming agents, one or more gas-generating agents, and pharmaceutically acceptable excipients into a pharmaceutically acceptable vehicle to form a suspension base; and
(v) dispersing the coated cores of step (iii) in the suspension base of step (iv) to obtain the gastroretentive extended release suspension composition.

A third aspect of the present invention provides a process for the preparation of a gastroretentive extended release suspension composition, wherein the process comprises the steps of:
(A) preparing a powder for suspension comprising the steps of:
   (i) preparing cores comprising an active ingredient and one or more pharmaceutically acceptable excipients;
   (ii) dissolving/dispersing a release-controlling polymer and one or more pharmaceutically acceptable coating additives in a suitable solvent;
   (iii) applying the coating composition of step (ii) over the cores of step (i);
   (iv) mixing one or more pharmaceutically acceptable excipients with the coated cores of step (iii) to obtain the powder for suspension;
(B) preparing a suspension base by dissolving/dispersing one or more osmogents, one or more gel-forming agents, one or more gas-generating agents, and pharmaceutically acceptable excipients into a pharmaceutically acceptable vehicle; and
(C) reconstituting the powder for suspension of step (A) with the suspension base of step (B) to obtain the gastroretentive extended release suspension composition.

A fourth aspect of the present invention provides a process for the preparation of a gastroretentive extended release suspension composition, wherein the process comprises the steps of:
(A) preparing a powder for suspension comprising the steps of:
   (i) preparing cores comprising an active ingredient and one or more pharmaceutically acceptable excipients;
   (ii) dissolving/dispersing a release-controlling polymer and one or more pharmaceutically acceptable coating additives in a suitable solvent;
   (iii) applying the coating composition of step (ii) over the cores of step (i);
   (iv) mixing one or more osmogents, one or more gel-forming agents, one or more gas-generating agents, and one or more pharmaceutically acceptable excipients with the coated cores of step (iii) to obtain the powder for suspension; and
(B) reconstituting the powder for suspension of step (A) with a pharmaceutically acceptable vehicle to obtain the gastroretentive extended release suspension composition.

A fifth aspect of the present invention provides use of a suspension base for the preparation of a gastroretentive extended release suspension composition comprising an active ingredient, wherein the composition is characterized by having no substantial change in the in-vitro dissolution release profile upon storage for at least seven days.

According to one embodiment of the above aspect, the active ingredient is in the form of multiple cores coated with a release-controlling polymer.

According to another embodiment of the above aspect, the composition is characterized by having an osmolality ratio of at least about 1.

According to another embodiment of the above aspect, the suspension base is responsible for creating a hypertonic environment.

According to another embodiment of the above aspect, the suspension base comprises an osmogent.

According to another embodiment of the above aspect, the suspension base comprises a gel-forming agent and a gas-generating agent.

According to another embodiment of the above aspect, the composition is a suspension or a reconstituted powder for suspension.

The term “extended release,” as used herein, refers to the release profile of the active ingredient over an extended period of time, e.g., over a period of 4, 6, 8, 12, 24 hours, or more.

The term “gastroretentive,” as used herein, means that upon oral administration at least a portion of the composition remains in the stomach for a period that is longer than the normal emptying time from the stomach, i.e., longer than about 2 hours, particularly longer than about 3 hours, and more particularly longer than about 4, 6, 8, 10, 12, or 24 hours.

The term “hypertonic environment,” as used herein, means the suspension base has higher solute concentration which helps to generate high osmotic pressure such that there is no leaching of active ingredient from the extended released coated cores into the suspension base. In the present invention, the solutes are osmogents i.e., pharmaceutically acceptable inert water-soluble compounds that contribute towards generating hypertonic environment in the suspension base.

The term “osmolality ratio,” as used herein, means the ratio of osmolality of the external phase to the osmolality of the internal phase. The external phase herein means the suspension base without multiple coated cores of the active ingredient. The internal phase herein means the coated cores of the active ingredient. As the direct measurement of the osmolality of the internal phase i.e., coated cores is difficult, the osmolality of the internal phase herein, is represented as the osmolality of a solution which prevents significant leaching of the active ingredient from the coated cores into the solution. The leaching of the active ingredient from the coated cores is determined by the difference in the osmolalities across the coating layer and the absence of any significant leaching from the coated cores directs that the osmolality of the solution has become equal to the osmolality of the coated cores. The osmolality ratio of the extended release suspension compositions of present invention is at least about 1.

The term “osmolality,” as used herein, is expressed as number of moles of any water-soluble compound per kg of liquid phase. The liquid phase can be a suspension base or a solution. In the present invention, osmolality may be measured according to known methods, such as using a vapor pressure osmometer, a colloid osmometer, or a freezing point depression osmometer such as Osmomat 030 or Osmomat 3000, in particular by a freezing point depression osmometer.

In the gastroretentive extended release suspension compositions of the present invention, the osmolality of the suspension base remains equivalent upon storage for at least seven days. Particularly, the osmolality of the suspension base measured after one month remains equivalent to the osmolality of the suspension base measured as soon as practicable after preparation of the gastroretentive extended release suspension compositions. More particularly, the osmolality of the suspension base measured after three months or six months remains equivalent to the osmolality of the suspension base measured as soon as practicable after preparation of the gastroretentive extended release suspension compositions. The equivalent osmolality of the suspension base ensures that there is no leaching of the active ingredient from the coated cores into the suspension base.

The in-vitro dissolution release profile of the gastroretentive extended release suspension compositions of the present invention upon storage for at least seven days remains substantially similar to the initial in-vitro dissolution release profile obtained as soon as practicable after preparation of the gastroretentive extended release suspension compositions. Particularly, the in-vitro dissolution release profile of the gastroretentive extended release suspension compositions of the present invention upon storage at room temperature for at least one month remains substantially similar to initial in-vitro dissolution release profile obtained as soon as practicable after preparation of the gastroretentive extended release suspension compositions. More particularly, the in-vitro dissolution release profile of the gastroretentive extended release suspension compositions of the present invention upon storage at room temperature for at least three months remains substantially similar to initial in-vitro dissolution release profile obtained as soon as practicable after preparation of the gastroretentive extended release suspension compositions. More particularly, the in-vitro dissolution release profile of the gastroretentive extended release suspension compositions of the present invention upon storage for at least six months remains substantially similar to initial in-vitro dissolution release profile obtained as soon as practicable after preparation of the gastroretentive extended release suspension compositions. In the present invention, wide ranges of dissolution methodologies can be utilized for different active ingredients. These methodologies can be adopted to vary in hydrodynamic mechanism to simulate in-vivo conditions by using different dissolution apparatuses, volume of media, pH of media ranging from 1.0 to 7.5, any standard USP buffers with standard molarity, addition of surfactants, and or enzymes.

The gastroretentive extended release suspension composition of the present invention provides the consistent in-vivo release which ensures steady and predictable active ingredient release with minimal inter and intra subject variation throughout the shelf life of the composition.

The term “substantial,” as used herein refers to any value which lies within the range as defined by a variation of up to ±15 from the average value.

The term “stable,” as used herein, refers to chemical stability, wherein not more than 10% w/w of total related substances are formed on storage at 40° C. and 75% relative humidity (R.H.) or at 25° C. and 60% R.H. for a period of at least three months to the extent necessary for the sale and use of the composition.

The term “inert particle” as used herein, refers to a particle made from a sugar sphere also known as a non-pareil seed, a microcrystalline cellulose sphere, a dibasic calcium phosphate bead, a mannitol beads, a silica bead, a tartaric acid pellet, a wax based pellet, and the like.

The term “about,” as used herein, refers to any value which lies within the range defined by a variation of up to ±10% of the value.

The term “equivalent” as used herein, refers to any value which lies within the range defined by a variation of up to ±30% of the value.

The term “significant leaching,” as used herein means not more than 20% of the active ingredient is leached out from the coated cores into the solution.

The gastroretentive extended release suspension composition of the present invention may be in the form of a suspension or a reconstituted powder for suspension. The powder for suspension may comprise of coated cores of active ingredient or a mixture of coated cores of active ingredient, one or more osmogents, one or more gel forming agents and/or one or more gas generating agents, and pharmaceutically acceptable excipients. This powder for suspension may be reconstituted with a pharmaceutically acceptable vehicle or a suspension base to form a gastroretentive extended release suspension composition.

The term “suspension base,” as used herein, refers to a medium which is used to suspend the coated cores of the active ingredient or to reconstitute the extended release powder for suspension of the active ingredient. The suspension base comprises a pharmaceutically acceptable vehicle, one or more osmogents, and pharmaceutically acceptable excipients.

The pharmaceutically acceptable vehicle as used herein means aqueous vehicle. The diameter of the coated cores of the present invention ranges from about 10 μm to about 2000 μm, particularly from about 50 μm to about 1000 μm, and more particularly from about 150 μm to about 500 μm. The finer sizes of the coated cores help in avoiding grittiness in the mouth and are therefore are more acceptable. The cores of the present invention may comprise one or more pharmaceutically acceptable excipients such as binders, and optionally one more osmogents.

The active ingredient of the present invention includes any active ingredient which are mainly absorbed in the stomach, which have higher solubility in the stomach than in the intestine, which are poorly absorbed or degraded in the intestine, or which act locally in the stomach. The active ingredient may belong to a therapeutic category such as antidiabetic, antibiotic, antimicrobial, analgesic, antiallergic, antianxiety, antiasthmatic, anticancer, antidepressant, antiemetic, antiinflammatory, antiParkinson's, antiepileptic, antitussive, antiviral, immunosuppressant, diuretic, antimigraine, cardiovascular, sympathomimetic, cholinomemetic, adrenergic, antimuscarinic, antispasmodic active ingredients, and skeletal muscle relaxants. The active ingredient of the present invention can be present in the form of a free base, in the form of pharmaceutically acceptable salts, or prodrugs. Specific examples of active ingredients include but are not limited to, metformin, acyclovir, gabapentin, pregabalin, trimetazidine, feropenem, carbidopa, levodopa, methyldopa, verapamil, propranolol, carvedilol, atenolol, albuterol, pirbuterol, nifedipine, nimodipine, nicardipine, amlodipine, prazosin, allopurinol, metoprolol, oxprenolol, baclofen, sumatriptan, benazepril, enalapril, lisinopril, captopril, quinapril, moxipril, indolapril, olindapril, retinapril, spirapril, cilazapril, perindopril, ramipril, zofenopril, fosinopril, nitrofurantoin, valacyclovir, azithromycin, inosine, didanosine, pranobex, tribavirin, vidarabine, simvastatin, pravastatin, atorvastatin, lovastatin, selegiline, midazolam, lithium carbonate, cimetidine, ranitidine, famotidine, nizatidine, bifentidine, nifentidine, roxatidine, antacids (such as magnesium carbonate, aluminum carbonate, aluminum hydroxide, magnesium oxide and sucralfate), carbenoloxalone, misoprostol, pirenzepine, telenzepine, bismuth salts, metronidazole, ciprofloxacin, amoxicillin, cephalexin, ascorbic acid, folic acid, vitamin E, furosemide, topiramide, hydrochlorothiazide, orlistat, alfuzosin, amiodarone, celiprolol, chlorprothixene, cyproheptadine, dasatinib, desipramine, dipyridamole, disopyramide, donepezil, haloperidol, hydralazine, imatinib, etoposide, lidocaine, maprotiline, mifepristone, nilotinib, orphenadrine, paliperidone, pramoxine, procyclidine, promethazine, propafenone, silodosin, terazosin, thioridazine, trihexyphenidyl, trimethoprim, progesterone, tacrolimus, estradiol, budesonide, norgestrel, alendronate, betamethasone, biperiden, ergotamine, estramustine, melphalan, methsuximide, mitotane, phenoxybenzamine, amiloride, azathioprine, bromocriptine, chlorpropamide, chlorthalidone, cortisone, danazol, diflunisal, fenofibrate, fludrocortisone, isradipine, loperamide, maprotiline, methyltestosterone, nabumetone, nortriptyline, oxcarbazepine, piroxicam, probenecid, propylthiouracil, tamoxifen, triazolam, trihexyphenidyl, trimipramine, calcitonin, butoconazole, econazole, amrinone, aloxiprin, aminoglutethimide, astemizole, beclomethasone, bendrofluazide, bexafibrate, bezafibrate, bromazepan, busulfan, camptothecin, carbimazole, cinnarizine, cisapride, clavulanic acid, clioquinol, clofibrate, clotiazepam, cyclizine, darodipine, decoquinate, dexanabinol, dextropropyoxyphene, dicoumarol, dihydrocodeine, domperidone, ethopropazine, fenbufen, fenfluramine, flunarizine, flunitrazepam, fluopromazine, flupenthixol, gliclazide, imidopril, lysuride, mazindol, meclofenamic acid, mefenamic acid, mepenzolate, mesalazine, methaqualone, methysergide, mianserin, neostigmine, nicoumalone, nitrazepam, norethisterone, oxprenolon, oxyphencyclimine, paramethadione, phenindione, phenylbutazone, pizotifen, probucol, propicillin, pyrantel, sulphadiazine, sulphafurazole, sulphamerazine, sulphapyridine, sulphasalazine, sulphinpyrazone, sulpiride, terfenadine, zopiclone, zaleplon, calcium, iron, lithium carbonate or citrate, calcium carbonate or citrate, riboflavin, captopril, pirbuterol, bismuth subsalicylate, bismuth subcitrate, misoprostol, 5-fluorouracil, doxorubicin, mitomycin, semustine, cisplatin, methotrexate, clarithromycin, methylnaltrexone, abacavir sulfate, lamivudine, zidovudine, acetazolamide, acetaminophen, albendazole, amoxicillin/clavulanate potassium, amprenavir, artesunate, atovaquone, proguanil hydrochloride, atracurium besylate, barium sulfate, beclomethasone dipropionate, betamethasone valerate, bismuth subsalicylate, bupropion, carbamazepine, caspofungin acetate, cefaclor, cefazolin, ceftazidime, cefuroxime, chlorambucil, chloroquin, chlorpromazine, clobetasol propionate, co-trimoxazole, dextroamphetamine, dioxin, dihydroxyartemisinin, doxycycline, epoprostenol, fluticasone propionate, glitazones, hydrotalcite, hydrocodone, hydrochlorothiazide, triamterene, lamotrigine, lithium carbonate, lomefloxacine, losartan potassium, mercaptopurine, mefloquine mesalazine, morphine, mupirocin calcium cream, nabumetone, naratriptan, norfloxacin, ofloxacine, ondansetron hydrochloride, oxiconazole nitrate, oxycodone, paroxetine hydrochloride, pefioxacine, piroxicam, prazodin, prochlorperazine, procyclidine hydrochloride, pyrimethamine, repaglinide, rofecoxib, ropinirole hydrochloride, rosiglitazone maleate, salmeterol, fluticasone propionate, sodium bicarbonate, spironolactone, succinylcholine chloride, tapentadol, thioguanine, topotecan hydrochloride, tramadol, tranylcypromine sulfate, sodium oxybate, isotretinoin, guaifenesin, dexmethylphenidate, methylphenidate, ranolazine, or trifluoperazine.

The dose of any active ingredient depends upon the individual active ingredient used in the gastroretentive extended release suspension compositions of the present invention. Further, the gastroretentive extended release suspension compositions of the present invention permit ready dose titration, i.e., adjusting the dose of the active ingredient based on recommended dose range and frequency until the desired therapeutic effect is achieved.

The gastroretentive extended release suspension compositions of the present invention may further include an immediate release component of the active ingredient to have a biphasic or pulsatile type of release. The immediate release component may be present in the form a powder, a pellet, a bead, a spheroid, a granule, or the like. Alternatively, the immediate release component may be present in the form of an immediate release coating over the coated cores. The immediate release component may help in providing an immediate therapeutic effect which could be subsequently followed by an extended therapeutic effect over a longer duration of time. Depending upon the type of polymer and percentage weight gain of the coating, the lag between the two phases can be adjusted to get the desired release profile.

The gastroretentive extended release suspension composition of the present invention may comprise two or more different active ingredients with different type of release profiles. One of the active ingredients provides the extended release, whereas another active ingredient may provide the immediate release or the extended release.

The gastroretentive extended release suspension composition of the present invention may further comprise two or more incompatible active ingredients present in a single composition. One of the active ingredients would be present in the form of coated cores providing the extended release and another incompatible active ingredient may be present in the form of a powder, a pellet, a bead, a spheroid, or a granule providing the immediate release or the extended release.

The gastroretentive extended release suspension compositions of the present invention are homogenous and delivers desired dose of the active ingredient in every use without any risk of overdosing or underdosing.

The gastroretentive extended release suspension composition of the present invention has a pH ranging from about 2 to about 10.

The release-controlling polymers used to form the extended release coating are selected from the group comprising a pH-dependent polymer, a pH-independent polymer, and mixtures thereof.

Suitable examples of pH-dependent polymers are selected from the group comprising acrylic copolymers such as methacrylic acid and methyl methacrylate copolymers, e.g., Eudragit® L 100 and Eudragit® S 100, methacrylic acid and ethyl acrylate copolymers, e.g., Eudragit® L 100-55 and Eudragit® L 30 D-55, dimethylaminoethyl methacrylate and butyl methacrylate and methyl methacrylate copolymer e.g., Eudragit® E 100, Eudragit® E PO, methyl acrylate and methacrylic acid and octyl acrylate copolymers, styrene and acrylic acid copolymers, butyl acrylate and styrene and acrylic acid copolymers, and ethylacrylate-methacrylic acid copolymer; cellulose acetate phthalate; cellulose acetate succinates; hydroxyalkyl cellulose phthalates such as hydroxypropylmethyl cellulose phthalate; hydroxyalkyl cellulose acetate succinates such as hydroxypropylmethyl cellulose acetate succinate; vinyl acetate phthalates; vinyl acetate succinate; cellulose acetate trimelliate; polyvinyl derivatives such as polyvinyl acetate phthalate, polyvinyl alcohol phthalate, polyvinyl butylate phthalate, and polyvinyl acetoacetal phthalate; zein; shellac; or mixtures thereof.

Suitable examples of pH-independent polymers are selected from the group comprising cellulosic polymers such as ethyl cellulose, methyl cellulose, hydroxyethyl cellulose, hydroxypropyl cellulose, hydroxyethylmethyl cellulose, hydroxypropylmethyl cellulose, and carboxy methylcellulose; acrylic copolymers such as methacrylic acid copolymers, e.g., Eudragit® RS, Eudragit® RL, Eudragit® NE 30 D; cellulose acetate; polyethylene derivatives e.g., polyethylene glycol and polyethylene oxide; polyvinyl alcohol; polyvinyl acetate; gums e.g., guar gum, locust bean gum, tragacanth, carrageenan, alginic acid, gum acacia, gum arabic, gellan gum, and xanthan gum; triglycerides; waxes, e.g., Compritol®, Lubritab®, and Gelucires®; lipids; fatty acids or their salts/derivatives; a mixture of polyvinyl acetate and polyvinyl pyrrolidone, e.g., Kollidon® SR; or mixtures thereof.

The term “osmogent,” as used herein, refers to all pharmaceutically acceptable inert water-soluble compounds that can imbibe or dissolve in water and/or aqueous biological fluids. Suitable examples of osmogents or pharmaceutically acceptable inert water-soluble compounds are selected from the group comprising carbohydrates such as xylitol, mannitol, sorbitol, arabinose, ribose, xylose, glucose, fructose, mannose, galactose, sucrose, maltose, lactose, dextrose and raffinose; water-soluble salts of inorganic acids such as magnesium chloride, magnesium sulfate, potassium sulfate, lithium chloride, sodium chloride, potassium chloride, lithium hydrogen phosphate, sodium hydrogen phosphate, potassium hydrogen phosphate, lithium dihydrogen phosphate, sodium dihydrogen phosphate, potassium dihydrogen phosphate, and sodium phosphate tribasic; water-soluble salts of organic acids such as sodium acetate, potassium acetate, magnesium succinate, sodium benzoate, sodium citrate, and sodium ascorbate; water-soluble amino acids such as glycine, leucine, alanine, methionine; urea or its derivatives; propylene glycol; glycerin; polyethylene oxide; xanthan gum; hydroxypropylmethyl cellulose; or mixtures thereof. Particularly, the osmogents used in the present invention are xylitol, mannitol, glucose, lactose, sucrose, and sodium chloride.

The term “gel-forming agent,” as used herein, refers to an agent which forms a gel upon contact with water. Preferred gel-forming agents are selected from the group comprising gums, e.g., alginic acid or its salts, xanthan gum, guar gum, locust bean gum, tragacanth, carrageenan, gum acacia, gum arabic, and gellan gum; saccharides, e.g., pectin or its derivatives, dextrin, polydextrin, dextran, polygalacturonic acid, xylan, arabinoxylan, and arabinogalactan; starch and starch based polymers or derivatives such as pregelatinized starch; cellulosic polymers, e.g., hydroxypropyl cellulose, hydroxymethyl cellulose, hydroxyethyl cellulose, methyl cellulose, sodium carboxymethyl cellulose, and hydroxypropyl methyl cellulose; a polyvinyl derivative e.g., polyvinyl alcohol and polyvinyl pyrrolidone; chitosan; carboxyvinyl polymers (such as those available under the trade name Carbopol®); a polyethylene derivative, e.g., polyethylene glycol and polyethylene oxide; a protein, e.g., gelatin; and mixtures thereof. In the present invention, the amount of gel-forming agent may range from about 0.1% to about 40% w/w based on the total weight of the composition.

The term “gas-generating agent,” as used herein, refers to agent that generate non-toxic gas upon contact with gastric fluid. Preferred gas-generating agents are selected from the group comprising carbonates or bicarbonates of an alkali or alkaline earth metal such as potassium carbonate or potassium bicarbonate, sodium carbonate or sodium bicarbonate, calcium carbonate, sodium glycine carbonate, magnesium carbonate, and aluminum carbonate; and sulfites such as sodium sulfite, sodium bisulfate, and sodium metabisulfite. These salts may be used alone or in combination with an acid source as a gas-generating couple. The acid source may be an edible organic acid, a salt of an edible organic acid, or a mixture thereof. Preferred organic acids include citric acid, malic acid, succinic acid, tartaric acid, fumaric acid, ascorbic acid, glutamic acid, and mixtures thereof. In the present invention, the amount of gel-forming agent may range from about 0.1% to about 40% w/w based on the total weight of the composition.

In the present invention, a gel-forming agent or a gas-generating agent may be present alone for achieving gastroretention. Alternatively, a combination of both gel-forming agent and gas-generating agent may be used to form a raft system to achieve gastroretention. In particular, the present invention uses a raft system. In this system, the gel forming agent forms a gel upon contact with gastric fluids and the gas generating agent forms a gas upon contact with acidic components present in the stomach. The gel thus formed entraps the gas and starts floating on the stomach contents. This raft system ensures immediate and complete entrapment of the multiple extended release coated cores of the active ingredient to provide the desired extended release of the active ingredient. This raft system may incorporate a processed starch such as pregelatinized starch and/or a dextrin in addition to the gel-forming and gas generating agents in order to further improve the strength and integrity of the system. The present invention may also involve the use of low density pellets to achieve the gastroretention.

The term “pharmaceutically acceptable excipients,” as used herein, refers to excipients that are routinely used in pharmaceutical compositions. The pharmaceutically acceptable excipients may comprise metal ion sources, glidants, sweeteners, suspending agents, anti-caking agents, wetting agents, preservatives, buffering agents, flavoring agents, anti-oxidants, chelating agents, or combinations thereof.

For gel-forming agents that exhibit ionotropic gelation, such as sodium alginate or pectin, a metal ion sources such as divalent or trivalent metal ions may also be included, to act as cross-linking agents to prepare rafts of higher strength. Suitable sources of divalent metal ions such as calcium ions are those derived from the carbonate, lactate, chloride, gluconate, phosphate, hydrogen phosphate, sulphate, tartrate, and citrate salts. Suitable sources of trivalent metal ions such as aluminum ions are derived from the carbonate, lactate, glycinate, or phosphate salts, or from aluminum magnesium carbonate hydroxide, magaldrate, aluminum sodium carbonate hydroxide, and aluminum sodium silicate.

Suitable glidants are selected from the group comprising silica, calcium silicate, magnesium silicate, colloidal silicon dioxide, cornstarch, talc, stearic acid, magnesium stearate, calcium stearate, sodium stearyl fumarate, hydrogenated vegetable oil, and mixtures thereof.

Suitable sweeteners are selected from the group comprising saccharine or its salts such as sodium, potassium, or calcium, cyclamate or its salt, aspartame, alitame, acesulfame or its salt, stevioside, glycyrrhizin or its derivatives, sucralose, or mixtures thereof.

Suitable suspending agents are selected from the group comprising cellulose derivatives such as co-processed spray dried forms of microcrystalline cellulose and carboxymethyl cellulose sodium, hydroxypropyl cellulose, hydroxyethyl cellulose, hydroxypropylmethyl cellulose, methylcellulose, carboxymethyl cellulose and its salts/derivatives, and microcrystalline cellulose; carbomers; gums such as locust bean gum, xanthan gum, tragacanth gum, arabinogalactan gum, agar gum, gellan gum, guar gum, apricot gum, karaya gum, sterculia gum, acacia gum, gum arabic, and carrageenan; pectin; dextran; gelatin; polyethylene glycols; polyvinyl compounds such as polyvinyl acetate, polyvinyl alcohol, and polyvinyl pyrrolidone; sugar alcohols such as xylitol and mannitol; colloidal silica; and mixtures thereof. Co-processed spray dried forms of microcrystalline cellulose and carboxymethyl cellulose sodium have been marketed under the trade names Avicel® RC-591 and Avicel® CL-611.

Suitable anti-caking agents are selected from the group comprising colloidal silicon dioxide, tribasic calcium phosphate, powdered cellulose, magnesium trisilicate, starch, and mixtures thereof.

Suitable wetting agents are selected from the group comprising anionic, cationic, nonionic, and zwitterionic surfactants, and combinations thereof. Preferred wetting agents are sodium lauryl sulphate; cetrimide; polyethylene glycols; polyoxyethylene-polyoxypropylene block copolymers such as poloxamers; polyglycerin fatty acid esters such as decaglyceryl monolaurate and decaglyceryl monomyristate; sorbitan fatty acid esters such as sorbitan monostearate; polyoxyethylene sorbitan fatty acid esters such as polyoxyethylene sorbitan monooleate; polyethylene glycol fatty acid esters such as polyoxyethylene monostearate; polyoxyethylene alkyl ethers such as polyoxyethylene lauryl ether; polyoxyethylene castor oil; and mixtures thereof.

Suitable preservatives are selected from the group comprising parabens such as methyl paraben and propyl paraben; sodium benzoate; and mixtures thereof.

Suitable buffering agents are selected from the group comprising citric acid, sodium citrate, sodium phosphate, potassium citrate, acetate buffer, and mixtures thereof.

Suitable flavoring agents are selected from FDA-approved flavoring agents, including peppermint, grapefruit, orange, lime, lemon, mandarin, pineapple, strawberry, raspberry, mango, passion fruit, kiwi, apple, pear, peach, apricot, cherry, grape, banana, cranberry, blueberry, black currant, red currant, gooseberry, lingon berries, cumin, thyme, basil, camille, valerian, fennel, parsley, chamomile, tarragon, lavender, dill, bargamot, salvia, aloe vera balsam, spearmint, eucalyptus, and combinations thereof.

Suitable anti-oxidants are selected from the group comprising butylated hydroxytoluene (BHT), butylated hydroxyanisole (BHA), sodium metabisulfite, ascorbic acid, propyl gallate, thiourea, tocopherols, beta-carotene, and mixtures thereof.

Suitable chelating agents are selected from the group comprising ethylenediamine tetraacetic acid or derivatives/salts thereof, e.g., disodium edetate; dihydroxyethyl glycine; glucamine; acids, e.g., citric acid, tartaric acid, gluconic acid, and phosphoric acid; and mixtures thereof.

Suitable binders are selected from the group comprising polyvinyl pyrrolidone, starch, pregelatinized starch, hydroxypropylmethyl cellulose, hydroxyethyl cellulose, methyl cellulose, sodium carboxymethyl cellulose, gums, acrylate polymers, and mixtures thereof.

The cores of the present invention comprising the active ingredient can be prepared by any method known in the art, e.g., extrusion-spheronoization, wet granulation, dry granulation, hot-melt extrusion granulation, spray drying, and spray congealing. Alternatively, the active ingredient can be layered onto an inert particle to form the core.

Further, the active ingredient particles can be directly coated with a release-controlling polymer to form the microparticles or microcapsules. The microparticles or microcapsules can be prepared by a process of homogenization, solvent evaporation, coacervation phase separation, spray drying, spray congealing, polymer precipitation, or supercritical fluid extraction.

The gastroretentive extended release suspension compositions of the present invention may further comprise one or more seal coating layers which may be applied before and/or after the functional coating layer. The seal coating layer may comprise of one or more film forming polymers and coating additives.

Examples of film-forming polymers include ethylcellulose, hydroxypropyl methylcellulose, hydroxypropylcellulose, methylcellulose, carboxymethyl cellulose, hydroxymethylcellulose, hydroxyethylcellulose, cellulose acetate, hydroxypropyl methylcellulose phthalate, cellulose acetate phthalate, cellulose acetate trimellitate; waxes such as polyethylene glycol; methacrylic acid polymers such as Eudragit®. Alternatively, commercially available coating compositions comprising film-forming polymers marketed under various trade names, such as Opadry® may also be used.

The coating additives used in the present invention are selected from the group comprising plasticizers, opacifiers, anti-tacking agents, coloring agents, and combinations thereof.

Suitable plasticizers are selected from the group comprising triethyl citrate, dibutyl sebacate, triacetin, acetylated triacetin, tributyl citrate, glyceryl tributyrate, diacetylated monoglyceride, rapeseed oil, olive oil, sesame oil, acetyl tributyl citrate, acetyl triethyl citrate, glycerin, sorbitol, diethyl oxalate, diethyl phthalate, diethyl malate, diethyl fumarate, dibutyl succinate, diethyl malonate, dioctyl phthalate, and combinations thereof.

Suitable opacifiers are selected from the group comprising titanium dioxide, manganese dioxide, iron oxide, silicon dioxide, and combinations thereof.

Suitable anti-tacking agents are selected from the group comprising talc, magnesium stearate, calcium stearate, stearic acid, silica, glyceryl monostearate, and combinations thereof.

Suitable coloring agents are selected from the group consisting of FD&C (Federal Food, Drug and Cosmetic Act) approved coloring agents; natural coloring agents; natural juice concentrates; pigments such as iron oxide, titanium dioxide, and zinc oxide; and combinations thereof.

Coating may be performed by applying the coating composition as a solution/suspension/blend using any conventional coating technique known in the art, such as spray coating in a conventional coating pan, fluidized bed processor, dip coating, or compression coating. The percentage of the coating build-up shall be varied depending on the required extended release.

Suitable solvents used for granulation or for forming a solution or dispersion for coating are selected from the group consisting of water, ethanol, methylene chloride, isopropyl alcohol, acetone, methanol, and combinations thereof.

The gastroretentive extended release suspension compositions of the present invention may be packaged in a suitable package such as a bottle. The powder for suspension may be packaged in a suitable package such as a bottle or a sachet. Further, the sachet can be filled as a unit dose or a multidose sachet. The present invention further includes a co-package or a kit comprising two components, wherein one package or one component comprises a powder for suspension and another package or another component comprises a pharmaceutically acceptable vehicle.

The invention is further illustrated by the following examples, which are for illustrative purposes only and should not be construed as limiting the scope of the invention in any way.

### EXAMPLES

### Example 1

| | |
|---|---|
| Ingredients | Quantity (mg/mL) |

Core

| | |
|---|---|
| Metformin hydrochloride | 80.00 |
| Microcrystalline cellulose spheres | 56.00 |
| Hydroxypropylmethyl cellulose | 4.00 |
| Purified water | q.s. |

Extended Release Coating

| | |
|---|---|
| Ethyl cellulose | 54.65-61.48 |
| Dibutyl sebacate | 1.35-1.52 |
| Acetone | q.s. |
| Purified water | q.s. |
| Total Weight of Extended Release | 196.00 mg |
| Beads | |
| Metformin hydrochloride | 20.00 |
| Xylitol | 450.00 |
| Sodium alginate | 37.5 |
| Pregelatinized starch | 24.00 |
| Sodium bicarbonate | 20.00 |
| Calcium carbonate | 12.00 |
| Methyl paraben | 1.80 |
| Propyl paraben | 0.20 |
| Strawberry flavor | 2.000 |
| Sucralose | 0.50 |
| Colloidal silicon dioxide | 5.00 |

Vehicle

| | |
|---|---|
| Purified water | q.s. to 1 mL |

### Procedure:

1. Metformin hydrochloride and hydroxypropylmethyl cellulose were dissolved in purified water.
2. Microcrystalline cellulose spheres were coated with the solution of step 1.
3. Ethyl cellulose and dibutyl sebacate were dispersed in a mixture of acetone and purified water.
4. The beads of step 2 were coated with the coating dispersion of step 3.
5. Metformin hydrochloride, xylitol, sodium alginate, pregelatinized starch, sodium bicarbonate, calcium carbonate, methyl paraben, propyl paraben, strawberry flavor, sucralose, and colloidal silicon dioxide were mixed.

6. The coated beads of step 4 were mixed with the mixture of step 5 to obtain a powder for suspension.

7. The powder for suspension of step 6 is reconstituted with required amount of purified water when required to form the gastroretentive extended release suspension composition.

### In-Vitro Studies

The gastroretentive extended release suspension composition prepared as per Example 1 was stored at room temperature for 30 days. The in-vitro dissolution was determined at 0 and 30 days using USP type II apparatus at 100 rpm, in 1000 mL of phosphate buffer with pH 6.8 at 37° C. The results of the release studies are represented in Table 1.

TABLE 1
Percentage (%) of the In-Vitro Metformin Release in USP
Type II Apparatus (Media: Phosphate Buffer, pH 6.8,
1000 mL, and 100 rpm)

Number of Days

| | |
|---|---|
| 0 | 30 |

| | |
|---|---|
| | Percentage of |
| | Metformin |
| Time (hours) | Release |

| | | |
|---|---|---|
| 0.5 | 22 | 21 |
| 1 | 28 | 28 |
| 2 | 63 | 64 |
| 3 | 80 | 81 |
| 4 | 88 | 88 |
| 5 | 92 | 93 |
| 6 | 95 | 94 |
| 8 | 98 | 99 |
| 10 | 99 | 100 |
| 12 | 101 | 102 |

From the above in-vitro release data, it is evident that the gastroretentive extended release suspension composition prepared according to Example 1 provides the substantially similar in-vitro metformin release for 30 days.

The powder for suspension prepared as per Example 1 (till step 6) was kept for one month at accelerated conditions i.e., 40° C./75% R.H. After one month, the powder for suspension was reconstituted with required amount of purified water and this gastroretentive extended release suspension composition was kept for 30 days at room temperature. The in-vitro dissolution was determined at 0, 30 days using USP type II apparatus at 100 rpm, in 1000 mL of phosphate buffer with pH 6.8 at 37° C. The results of the release studies are represented in Table 2.

TABLE 2
Percentage (%) of the In-Vitro Metformin Release in USP
Type II Apparatus (Media: Phosphate Buffer, pH 6.8,
1000 mL, and 100 rpm)

Number of
Days After
Reconstitution

| | |
|---|---|
| 0 | 30 |

| | |
|---|---|
| | Percentage of |
| | Metformin |
| Time (hours) | Release |

| | | |
|---|---|---|
| 0.5 | 21 | 21 |
| 1 | 29 | 29 |
| 2 | 64 | 64 |
| 3 | 79 | 81 |
| 4 | 85 | 87 |
| 5 | 89 | 91 |
| 6 | 91 | 93 |
| 8 | 94 | 97 |
| 10 | 93 | 99 |
| 12 | 98 | 100 |

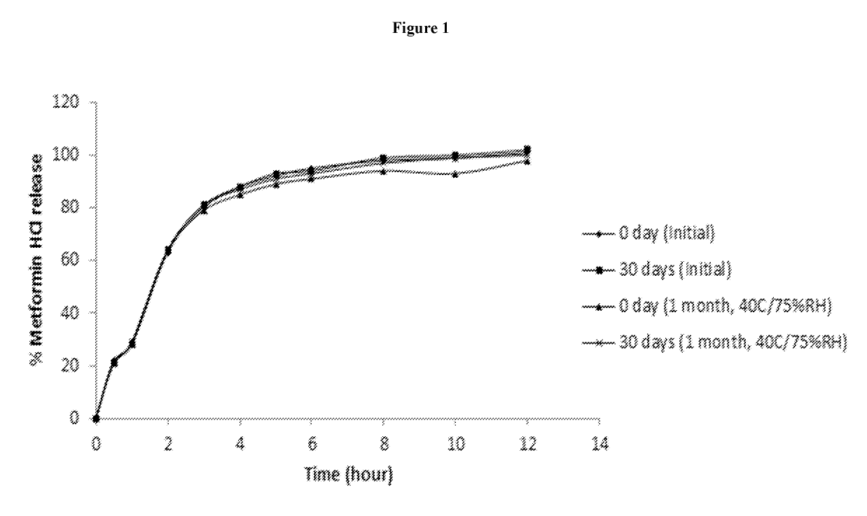

From the above data, it is clear that the extended release powder prepared according to Example 1 stored at accelerated condition for one month, upon reconstitution and storage for 30 days at room temperature provides substantially similar in-vitro metformin release for 30 days. The results are presented in FIG. 1.

### Osmolality Measurement of the Gastroretentive Extended Release Suspension

The gastroretentive extended release powder for suspension prepared according to Example 1 was reconstituted with required amount of purified water. This suspension was shaken manually for at least 20 minutes. This suspension was then filtered and diluted with purified water and the osmolality was measured using Osmomat 030-D.

The osmolality of the suspension base was found to be 4.320 osmol/kg of the suspension base on day 0.

The osmolality of the suspension base was found to be 4.476 osmol/kg of the suspension base on day 7.

It is evident from the above data that the osmolality of the suspension base of the gastroretentive extended release suspension composition remains equivalent for seven days.

### Dose Uniformity Data

The gastroretentive extended release suspension equivalent to 100 mL was prepared according to formula given in Example 1. This suspension was shaken manually for at least 20 minutes and then ten 7.5 mL samples were taken with a graduated syringe. The metformin content of each sample is determined by HPLC method [Inertsil ODS column (250×4.6 mm, 5 μm); mobile phase-buffer (pH 3.5):acetonitrile (95:5 v/v); flow rate of 1.5 mL/min; UV detection at 233 nm] The results are shown in Table 3.

TABLE 3
Metformin Content (%) For Each 7.5 mL of Suspension

| Sample | Metformin content (%) for each |
|---|---|
| Number | 7.5 mL of suspension |
| 1 | 97.0 |
| 2 | 96.4 |
| 3 | 96.7 |
| 4 | 96.5 |
| 5 | 96.2 |
| 6 | 97.4 |
| 7 | 96.3 |
| 8 | 95.8 |
| 9 | 95.9 |
| 10 | 95.5 |
| Mean value | 96.4 |

Form the above data, it is evident that the gastroretentive extended release suspension composition prepared according to Example 1 is homogeneous.

### Assay Data

The assay for the gastroretentive extended release suspension composition prepared as per Example 1 was determined at 0 day and after storage at room temperature for 30 days. The powder for suspension prepared as per Example 1 (till step 6) was kept for one month at accelerated conditions i.e., 40° C./75% R.H. After one month, the powder for suspension was reconstituted with required amount of purified water and then assay was determined at 0 day and after storage at room temperature for 30 days. The assay of metformin was determined by HPLC method [Inertsil ODS column (250×4.6 mm, 5 μm); mobile phase-buffer (pH 3.5):acetonitrile (95:5 v/v); flow rate of 1.5 mL/min; UV detection at 233 nm]. The results are shown in Table 4.

TABLE 4
Assay for Metformin

Assay (%)
for metformin

| Condition | 0 day | 30 days |
|---|---|---|
| Initial | 101.0 | 101.4 |
| 1 month | 100.0 | 98.7 |
| (40° C./75% R.H) | | |

It is evident from the above data that the gastroretentive extended release suspension composition prepared as per Example 1 is stable.

### Osmolality Measurement of the External Phase

Metformin hydrochloride, xylitol, sodium alginate, pregelatinized starch, sodium bicarbonate, calcium carbonate, methyl paraben, propyl paraben, strawberry flavor, sucralose, and colloidal silicon dioxide were mixed as given in step 5 of Example 1. This powder was reconstituted with required amount of purified water. This suspension was then filtered and diluted with purified water and the osmolality was measured using an Osmomat 030-D.

The osmolality of the suspensions base was found to be 4.236 osmol/kg of the suspension base.

### Osmolality Measurement of the Internal Phase

Various solutions having various concentrations of osmogent (sodium chloride) were prepared as per Examples 1A-1F. The osmolalities of these solutions were measured using an Osmomat 030-D.

| Ingredient | Example 1A | Example 1B | Example 1C | Example 1D | Example 1E | Example 1F |
|---|---|---|---|---|---|---|
| Sodium | 30.00 | 60.00 | 120.00 | 180.00 | 240.00 | 300.00 |
| Chloride (mg) | | | | | | |
| Purified | q.s. to 1 mL | q.s. to 1 mL | q.s. to 1 mL | q.s. to 7.5 mL | q.s. to 1 mL | q.s. to 1 mL |
| water | | | | | | |
| Osmolality | 0.910 | 1.787 | 3.574* | 5.361* | 7.148* | 8.935* |
| (osmol/kg) | | | | | | |

*Extrapolated using values of dilute solutions

The coated beads of step 4 (61.48 mg of ethyl cellulose and 1.52 mg of dibutyl sebacate) were dispersed in different solutions as per Examples 1A-1F. These solutions were kept for seven days at room temperature. After seven days, each solution was analyzed by HPLC for metformin content. The results are represented in following Table 5.

TABLE 5
Effect of Osmolality on Metformin Leaching

| | Osmolality (osmol/kg) | Metformin |
|---|---|---|
| Example | of the solution | content (%) |
| 1A | 0.910 | 67.3 |
| 1B | 1.787 | 30.3 |
| 1C | 3.574* | 2.9 |
| 1D | 5.361* | 1.8 |
| 1E | 7.148* | 1.7 |
| 1F | 8.935* | 1.0 |

*Extrapolated using values of dilute solutions

From the above data, it is evident that the leaching of metformin from the coated beads into the solution was decreasing as the osmolality of the solution was increasing from Examples 1A-1F. The leaching is found to be significantly reduced from Example 1C onwards. The osmolality of Example 1C i.e., 3.574 is considered as osmolality of the internal phase.

### Osmolality Ratio 1.185

We claim:

1. A gastroretentive extended release suspension composition comprising an active ingredient and a suspension base, wherein the composition is characterized by having no substantial change in the in-vitro dissolution release profile upon storage for at least seven days.

2. The gastroretentive extended release suspension composition of claim 1, wherein the active ingredient is in the form of multiple cores coated with a release-controlling polymer.

3. The gastroretentive extended release suspension composition of claim 1, wherein the composition is characterized by having an osmolality ratio of at least about 1.

4. The gastroretentive extended release suspension composition of claim 1, wherein the suspension base is responsible for creating a hypertonic environment.

5. The gastroretentive extended release suspension composition of claim 1, wherein the suspension base comprises an osmogent.

6. The gastroretentive extended release suspension composition of claim 5, wherein the suspension base further comprises a gel-forming agent and a gas-generating agent.

7. The gastroretentive extended release suspension composition of claim 1, wherein the composition is a suspension or a reconstituted powder for suspension.

8. The gastroretentive extended release suspension composition of claim 2, wherein active ingredient is layered onto an inert particle to form the core.

9. The gastroretentive extended release suspension composition of claim 8, wherein the inert particle is selected from the group comprising a non-pareil seed, a microcrystalline cellulose sphere, a dibasic calcium phosphate bead, a mannitol bead, a silica bead, a tartaric acid pellet, or a wax based pellet.

10. The gastroretentive extended release suspension composition of claim 6, wherein the gel-forming agent is selected from group comprising alginic acid or its salts, xanthan gum, guar gum, locust bean gum, tragacanth, carrageenan, gum acacia, gum arabic, gellan gum, pectin or its derivatives, dextrin, polydextrin, dextran, polygalacturonic acid, xylan, arabinoxylan, arabinogalactan, starch, hydroxypropyl cellulose, hydroxymethyl cellulose, hydroxyethyl cellulose, methyl cellulose, sodium carboxymethyl cellulose, hydroxypropyl methyl cellulose, polyvinyl alcohol, polyvinyl pyrrolidone, chitosan, carboxyvinyl polymer, polyethylene glycol, polyethylene oxide, gelatin, and mixtures thereof.

11. The gastroretentive extended release suspension composition of claim 6, wherein the gas-generating agent is selected from the group comprising potassium carbonate, potassium bicarbonate, sodium carbonate, sodium bicarbonate, calcium carbonate, sodium glycine carbonate, magnesium carbonate, aluminum carbonate, sodium sulfite, sodium bisulfate, sodium metabisulfite, and mixtures thereof.

12. The gastroretentive extended release suspension composition of claim 2, wherein the release-controlling polymer is selected from the group comprising a pH-dependent polymer, a pH-independent polymer, or mixtures thereof.

13. The gastroretentive extended release suspension composition of claim 12, wherein the pH-dependent polymer is selected form the group comprising acrylic copolymers such as methacrylic acid and methyl methacrylate copolymers, e.g., Eudragit® L 100 and Eudragit® S 100, methacrylic acid and ethyl acrylate copolymers, e.g., Eudragit® L 100-55 and Eudragit® L 30 D-55, dimethylaminoethyl methacrylate and butyl methacrylate and methyl methacrylate copolymer e.g., Eudragit® E 100, Eudragit® E PO, methyl acrylate and methacrylic acid and octyl acrylate copolymers, styrene and acrylic acid copolymers, butyl acrylate and styrene and acrylic acid copolymers, and ethylacrylate-methacrylic acid copolymer; cellulose acetate phthalate; cellulose acetate succinates; hydroxyalkyl cellulose phthalates such as hydroxypropylmethyl cellulose phthalate; hydroxyalkyl cellulose acetate succinates such as hydroxypropylmethyl cellulose acetate succinate; vinyl acetate phthalates; vinyl acetate succinate; cellulose acetate trimelliate; polyvinyl derivatives such as polyvinyl acetate phthalate, polyvinyl alcohol phthalate, polyvinyl butylate phthalate, and polyvinyl acetoacetal phthalate; zein; shellac; or mixtures thereof.

14. The gastroretentive extended release suspension composition of claim 12, wherein the pH-independent polymer is selected from the group comprising cellulosic polymers such as ethyl cellulose, methyl cellulose, hydroxyethyl cellulose, hydroxypropyl cellulose, hydroxyethylmethyl cellulose, hydroxypropylmethyl cellulose, and carboxy methylcellulose; acrylic copolymers such as methacrylic acid copolymers, e.g., Eudragit® RS, Eudragit® RL, Eudragit® NE 30 D; cellulose acetate; polyethylene derivatives e.g., polyethylene glycol and polyethylene oxide; polyvinyl alcohol; polyvinyl acetate; gums e.g., guar gum, locust bean gum, tragacanth, carrageenan, alginic acid, gum acacia, gum arabic, gellan gum, and xanthan gum; triglycerides; waxes, e.g., Compritol®, Lubritab®, and Gelucires®; lipids; fatty acids or their salts/derivatives; a mixture of polyvinyl acetate and polyvinyl pyrrolidone, e.g., Kollidon® SR; or mixtures thereof.

15. The gastroretentive extended release suspension composition of claim 5, wherein the osmogent is selected from the group comprising carbohydrates such as xylitol, mannitol, sorbitol, arabinose, ribose, xylose, glucose, fructose, mannose, galactose, sucrose, maltose, lactose, dextrose and raffinose; water-soluble salts of inorganic acids such as magnesium chloride, magnesium sulfate, potassium sulfate, lithium chloride, sodium chloride, potassium chloride, lithium hydrogen phosphate, sodium hydrogen phosphate, potassium hydrogen phosphate, lithium dihydrogen phosphate, sodium dihydrogen phosphate, potassium dihydrogen phosphate, and sodium phosphate tribasic; water-soluble salts of organic acids such as sodium acetate, potassium acetate, magnesium succinate, sodium benzoate, sodium citrate, and sodium ascorbate; water-soluble amino acids such as glycine, leucine, alanine, methionine; urea or its derivatives; propylene glycol; glycerin; or mixtures thereof.

16. The gastroretentive extended release suspension composition of claim 1, wherein the active ingredient is selected from the group comprising metformin, acyclovir, gabapentin, pregabalin, trimetazidine, feropenem, carbidopa, levodopa, methyldopa, verapamil, propranolol, carvedilol, atenolol, albuterol, pirbuterol, nifedipine, nimodipine, nicardipine, amlodipine, prazosin, allopurinol, metoprolol, oxprenolol, baclofen, sumatriptan, benazepril, enalapril, lisinopril, captopril, quinapril, moxipril, indolapril, olindapril, retinapril, spirapril, cilazapril, perindopril, ramipril, zofenopril, fosinopril, nitrofurantoin, valacyclovir, azithromycin, inosine, didanosine, pranobex, tribavirin, vidarabine, simvastatin, pravastatin, atorvastatin, lovastatin, selegiline, midazolam, lithium carbonate, cimetidine, ranitidine, famotidine, nizatidine, bifentidine, nifentidine, roxatidine, antacids (such as magnesium carbonate, aluminum carbonate, aluminum hydroxide, magnesium oxide and sucralfate), carbenoloxalone, misoprostol, pirenzepine, telenzepine, bismuth salts, metronidazole, ciprofloxacin, amoxicillin, cephalexin, ascorbic acid, folic acid, vitamin E, furosemide, topiramide, hydrochlorothiazide, orlistat, alfuzosin, amiodarone, celiprolol, chlorprothixene, cyproheptadine, dasatinib, desipramine, dipyridamole, disopyramide, donepezil, haloperidol, hydralazine, imatinib, etoposide, lidocaine, maprotiline, mifepristone, nilotinib, orphenadrine, paliperidone, pramoxine, procyclidine, promethazine, propafenone, silodosin, terazosin, thioridazine, trihexyphenidyl, trimethoprim, progesterone, tacrolimus, estradiol, budesonide, norgestrel, alendronate, betamethasone, biperiden, ergotamine, estramustine, melphalan, methsuximide, mitotane, phenoxybenzamine, amiloride, azathioprine, bromocriptine, chlorpropamide, chlorthalidone, cortisone, danazol, diflunisal, fenofibrate, fludrocortisone, isradipine, loperamide, maprotiline, methyltestosterone, nabumetone, nortriptyline, oxcarbazepine, piroxicam, probenecid, propylthiouracil, tamoxifen, triazolam, trihexyphenidyl, trimipramine, calcitonin, butoconazole, econazole, amrinone, aloxiprin, aminoglutethimide, astemizole, beclomethasone, bendrofluazide, bexafibrate, bezafibrate, bromazepan, busulfan, camptothecin, carbimazole, cinnarizine, cisapride, clavulanic acid, clioquinol, clofibrate, clotiazepam, cyclizine, darodipine, decoquinate, dexanabinol, dextropropyoxyphene, dicoumarol, dihydrocodeine, domperidone, ethopropazine, fenbufen, fenfluramine, flunarizine, flunitrazepam, fluopromazine, flupenthixol, gliclazide, imidopril, lysuride, mazindol, meclofenamic acid, mefenamic acid, mepenzolate, mesalazine, methaqualone, methysergide, mianserin, neostigmine, nicoumalone, nitrazepam, norethisterone, oxprenolon, oxyphencyclimine, paramethadione, phenindione, phenylbutazone, pizotifen, probucol, propicillin, pyrantel, sulphadiazine, sulphafurazole, sulphamerazine, sulphapyridine, sulphasalazine, sulphinpyrazone, sulpiride, terfenadine, zopiclone, zaleplon, calcium, iron, lithium carbonate or citrate, calcium carbonate or citrate, riboflavin, captopril, pirbuterol, bismuth subsalicylate, bismuth subcitrate, misoprostol, 5-fluorouracil, doxorubicin, mitomycin, semustine, cisplatin, methotrexate, clarithromycin, methylnaltrexone, abacavir sulfate, lamivudine, zidovudine, acetazolamide, acetaminophen, albendazole, amoxicillin/clavulanate potassium, amprenavir, artesunate, atovaquone, proguanil hydrochloride, atracurium besylate, barium sulfate, beclomethasone dipropionate, betamethasone valerate, bismuth subsalicylate, bupropion, carbamazepine, caspofungin acetate, cefaclor, cefazolin, ceftazidime, cefuroxime, chlorambucil, chloroquin, chlorpromazine, clobetasol propionate, co-trimoxazole, dextroamphetamine, dioxin, dihydroxyartemisinin, doxycycline, epoprostenol, fluticasone propionate, glitazones, hydrotalcite, hydrocodone, hydrochlorothiazide, triamterene, lamotrigine, lithium carbonate, lomefloxacine, losartan potassium, mercaptopurine, mefloquine mesalazine, morphine, mupirocin calcium cream, nabumetone, naratriptan, norfloxacin, ofloxacin, ondansetron hydrochloride, oxiconazole nitrate, oxycodone, paroxetine hydrochloride, pefioxacine, piroxicam, prazodin, prochlorperazine, procyclidine hydrochloride, pyrimethamine, repaglinide, rofecoxib, ropinirole hydrochloride, rosiglitazone maleate, salmeterol, fluticasone propionate, sodium bicarbonate, spironolactone, succinylcholine chloride, tapentadol, thioguanine, topotecan hydrochloride, tramadol, tranylcypromine sulfate, sodium oxybate, isotretinoin, guaifenesin, dexmethylphenidate, methylphenidate, ranolazine, or trifluoperazine

17. The gastroretentive extended release suspension composition of any of the preceding claims, wherein the composition further comprises one or more pharmaceutically acceptable excipients selected from the group comprising suspending agents, anti-caking agents, wetting agents, preservatives, buffering agents, flavoring agents, anti-oxidants, and chelating agents.

18. A process for the preparation of a gastroretentive extended release suspension composition according to claim 1, wherein the process comprises the steps of:
(i) preparing cores comprising an active ingredient and one or more pharmaceutically acceptable excipients;
(ii) dissolving/dispersing a release-controlling polymer and one or more pharmaceutically acceptable coating additives in a suitable solvent;
(iii) applying the coating composition of step (ii) over the cores of step (i);
(iv) dissolving/dispersing one or more osmogents, one or more gel-forming agents, one or more gas-generating agents, and pharmaceutically acceptable excipients into a pharmaceutically acceptable vehicle to form a suspension base; and
(v) dispersing the coated cores of step (iii) in the suspension base of step (iv) to obtain the gastroretentive extended release suspension composition

19. A process for the preparation of an gastroretentive extended release suspension composition according to claim 1, wherein the process comprises the steps of:
(A) preparing a powder for suspension comprising the steps of:
(i) preparing cores comprising an active ingredient and one or more pharmaceutically acceptable excipients;
(ii) dissolving/dispersing a release-controlling polymer and one or more pharmaceutically acceptable coating additives in a suitable solvent;
(iii) applying the coating composition of step (ii) over the cores of step (i);
(iv) mixing one or more pharmaceutically acceptable excipients with the coated cores of step (iii) to obtain the powder for suspension;
(B) preparing a suspension base by dissolving/dispersing one or more osmogents, one or more gel-forming agents, one or more gas-generating agents, and pharmaceutically acceptable excipients into a pharmaceutically acceptable vehicle; and
(C) reconstituting the powder for suspension of step (A) with the suspension base of step (B) to obtain the gastroretentive extended release suspension composition.

20. A process for the preparation of an gastroretentive extended release suspension composition according to claim 1, wherein the process comprises the steps of:
(A) preparing a powder for suspension comprising the steps of:
(i) preparing cores comprising an active ingredient and one or more pharmaceutically acceptable excipients;
(ii) dissolving/dispersing a release-controlling polymer and one or more pharmaceutically acceptable coating additives in a suitable solvent;
(iii) applying the coating composition of step (ii) over the cores of step (i);
(iv) mixing one or more osmogents, one or more gel-forming agents, one or more gas-generating agents, and one or more pharmaceutically acceptable excipients with the coated cores of step (iii) to obtain the powder for suspension; and
(B) reconstituting the powder for suspension of step (A) with a pharmaceutically acceptable vehicle to obtain the gastroretentive extended release suspension composition.

21. Use of a suspension base for the preparation of a gastroretentive extended release suspension composition comprising an active ingredient, wherein the composition is characterized by having no substantial change in the in-vitro dissolution release profile upon storage for at least seven days.

22. The use of the suspension base of claim 21, wherein the active ingredient is in the form of multiple cores coated with a release-controlling polymer.

23. The use of the suspension base of claim 21, wherein the composition is characterized by having an osmolality ratio of at least about 1.

24. The use of the suspension base of claim 21, wherein the suspension base is responsible for creating a hypertonic environment.

25. The use of the suspension base of claim 21, wherein the suspension base comprises an osmogent.

26. The use of the suspension base of claim 25, wherein the suspension base further comprises a gel-forming agent and a gas-generating agent.

27. The use of the suspension base of claim 21, wherein the composition is a suspension or a reconstituted powder for suspension.
